(19) United States
(12) Patent Application Publication
(10) Pub. No.: US 2018/0049989 A1
(43) Pub. Date: Feb. 22, 2018

(54) PHARMACEUTICAL COMPOSITION OF TIZANIDINE AND PROCESS FOR PREPARING THE SAME

(71) Applicant: Jubilant Generics Limited, Uttar Pradesh (IN)

(72) Inventors: Pankaj SONI, Haryana (IN); Ganesh Vinayak GAT, PUNE (IN); Ravikumar NITHIYANANDAM, Tamil Nadu (IN); Rashmi Ranjan PANDA, Uttar Pradesh (IN); Dinesh KUMAR, Chandigarh (IN)

(21) Appl. No.: 15/555,748

(22) PCT Filed: Mar. 4, 2016
(86) PCT No.: PCT/IN2016/050076

§ 371 Date: Sep. 5, 2017

(30) Foreign Application Priority Data
Mar. 5, 2015 (IN) 617/DEL/2015

(51) Int. Cl. A61K 9/48; A61K 31/433

(52) U.S. Cl. CPC A61K 9/4858; A61K 31/433

(57) ABSTRACT

The present invention relates to a solid oral pharmaceutical composition comprising an effective amount of Tizanidine or its pharmaceutically acceptable salts, esters, solvates, polymorphs, stereoisomers or mixtures thereof. The solid oral dosage form of the present invention is free of non-pareil seeds. The invention also relates to a process for the preparation of a pharmaceutical composition comprising an effective amount of Tizanidine wherein, the dosage form is free of non-pareil seeds.

### FIELD OF THE INVENTION

Present invention relates to a pharmaceutical composition comprising Tizanidine or its pharmaceutically acceptable salts, esters, solvates, polymorphs, stereoisomers or mixtures thereof. In particular, the present invention provides a solid oral pharmaceutical composition comprising Tizanidine, wherein the composition is free of non-pareil seeds.

### BACKGROUND OF THE INVENTION

Tizanidine is a centrally acting α2 adrenergic agonist. It possesses myotonolytic activity and is useful in the treatment of spasticity in patients with cerebral or spinal injury, muscle spasm and pain.

Tizanidine Hydrochloride is chemically described as 5-chloro-4-(2-imidazolin-2-ylamino)-2,1,3-benzothiadiazole monohydrochloride and is represented by the following formula:

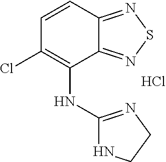 [SMILES: Clc1ccc2nsnc2c1NC1=NCCN1 ; Cl]

Tizanidine Hydrochloride is marketed as tablets and capsules under the brand names ZANAFLEX®. Inactive ingredients in the ZANAFLEX® capsules consist of Hydroxypropyl methylcellulose, silicon dioxide, sugar spheres, titanium dioxide, gelatin and colorants.

U.S. Pat. No. 3,843,668 discloses Tizanidine. Example 3 of the patent discloses preparation of crystals of Tizanidine from methanol having melting point of 221-223° C.

U.S. Pat. No. 4,053,617 discloses a method of treating spastic conditions which comprises administering to an animal in need of such treatment a therapeutically effective amount of Tizanidine Hydrochloride.

US 2014/0341985 discloses an immediate release multi-particulate Tizanidine oral dosage formulation which comprises a plurality of particles comprising from 2 mg to 12 mg of Tizanidine Hydrochloride and at least one pharmaceutically acceptable excipient. The particles comprise immediate release beads comprising Tizanidine layered over non-pareil seeds. Tizanidine Hydrochloride immediate release multi-particulates are prepared by spraying a solution of Tizanidine Hydrochloride, hydroxypropyl methylcellulose and silicon dioxide in water over non-pareil seeds (Sugar spheres). Pelletization is an expensive and time consuming process which requires highly specialized equipment and trained personnel. Moreover, control of the manufacturing process during pelletization is difficult due to various challenges associated with drug layering and control of critical process parameters.

Tizanidine Hydrochloride is approved for oral administration at low doses of 2 to 6 mg. Low doses of Tizanidine Hydrochloride offers manufacturing challenges of ensuring acceptable content uniformity of each of the low dose units. The small amount of drug substance that is typically used for the manufacture of these low dose units must be evenly distributed in a powder blend. Presently marketed capsule formulation of Tizanidine Hydrochloride in the USA (ZANAFLEX®) is a multi-particulate formulation which comprises layering of Tizanidine Hydrochloride over non-pareil seeds. A need exists in the pharmaceutical art to develop a formulation of Tizanidine Hydrochloride which is free of multi-particulate systems. There exists a need in the pharmaceutical art to develop a simple, reproducible, and cost-effective manufacturing process for pharmaceutical composition of Tizanidine Hydrochloride capsules which also offers comparable formulation technical attributes with respect to ZANAFLEX® capsules.

The present inventors developed a simple, reproducible and cost-effective process for preparing pharmaceutical composition of Tizanidine Hydrochloride. Further, the pharmaceutical compositions prepared according to the manufacturing process of the present invention possess desirable formulation characteristics.

### SUMMARY OF THE INVENTION

One embodiment of the present invention relates to pharmaceutical compositions comprising Tizanidine or its pharmaceutically acceptable salts, esters, solvates, polymorphs, stereoisomers or mixtures thereof.

In another embodiment, the present invention provides a process for the preparation of pharmaceutical compositions of Tizanidine or its pharmaceutically acceptable salts, esters, solvates, polymorphs, stereoisomers or mixtures thereof.

Another embodiment of the present invention includes solid oral pharmaceutical compositions comprising Tizanidine Hydrochloride prepared by dry granulation or wet granulation process.

Another embodiment of the present invention includes solid oral pharmaceutical compositions comprising Tizanidine Hydrochloride prepared by wet granulation method.

Another embodiment of the present invention encompasses a pharmaceutical composition comprising Tizanidine Hydrochloride and a pharmaceutically acceptable excipient selected from at least one of diluent and optionally binder, disintegrant, surfactant, lubricant and glidant.

In another embodiment, the present invention includes a pharmaceutical composition comprising Tizanidine Hydrochloride, wherein D₉₀ is less than about 200 μm and D₅₀ is less than about 80 μm.

In yet another embodiment of the invention, the pharmaceutical compositions comprise about 2 mg to about 36 mg of Tizanidine Hydrochloride.

In another embodiment, the granules present in the pharmaceutical composition have bulk density above 0.3 g/cm³.

In another embodiment, the granules present in the pharmaceutical composition have tapped density above 0.4 g/cm³.

In another embodiment, at least 30% the granules present in the pharmaceutical composition have a diameter of about 850 to about 250 μm [ASTM (American Society for Testing Materials) standards #20-60 mesh sieve]. Preferably, at least 40% the granules present in the pharmaceutical composition have a diameter of about 850 to about 250 μm [ASTM (American Society for Testing Materials) standards #20-60 mesh sieve]. More preferably, at least 50% the granules present in the pharmaceutical composition have a diameter of about 850 to about 250 μm [ASTM (American Society for Testing Materials) standards #20-60 mesh sieve].

In another embodiment, pharmaceutical compositions of the present invention are placed inside capsule shell of size 00 to 5. Preferably, pharmaceutical compositions of the present invention are placed inside capsule shell of size 1 to 4.

In further embodiment, the present invention includes method of using the pharmaceutical composition comprising Tizanidine Hydrochloride in the management of spasticity.

### DETAILED DESCRIPTION OF THE INVENTION

The present invention can be more readily understood by reading the following detailed description of the invention and study of the included examples.

As used herein, the term “composition”, as in pharmaceutical composition, is intended to encompass a drug product comprising Tizanidine or its pharmaceutically acceptable salts, esters, solvates, polymorphs, stereoisomers or mixtures thereof, and the other inert ingredient(s) (pharmaceutically acceptable excipients). Such pharmaceutical compositions are synonymous with “formulation” and “dosage form”. Pharmaceutical compositions of the invention include, but are not limited to, granules, tablets (single layered tablets, multilayered tablets, mini tablets, bioadhesive tablets, caplets, matrix tablets, tablet within a tablet, mucoadhesive tablets, modified release tablets, orally disintegrating tablets, pulsatile release tablets, timed release tablets, delayed release, controlled release, extended release and sustained release tablets), granules, capsules (hard and soft or liquid filled soft gelatin capsules), pills, troches, sachets, powders, microcapsules, minitablets, tablets in capsules and microspheres, matrix compositions and the like. Preferably, the pharmaceutical composition refers to tablets and capsules. More preferably, the pharmaceutical composition refers to hard gelatin capsules.

Unless otherwise stated the weight percentages expressed herein are based on the final weight of the composition or formulation.

Bulk density, as used herein, refers to the ratio of the mass of an untapped powder sample and its volume including the contribution of the interparticulate void volume. Bulk density indicates mass of a powder material that can be filled in per unit volume. Preferably, granules present in the pharmaceutical composition have bulk density above 0.3 g/cm³.

Tapped density, as used herein, refers to the ratio of the mass of a tapped powder sample and its volume. Tapped density of granules is determined using Electrolab tap density tester (Model ETD 1020) where tapping is done at a rate of 500 to 1250 strokes per minute (Spm). Preferably, granules present in the pharmaceutical composition have tapped density above 0.4 g/cm³.

The present invention relates to pharmaceutical composition of Tizanidine or its pharmaceutically acceptable esters, salts, esters, solvates, polymorphs, stereoisomers or mixtures thereof. Preferably, salt of Tizanidine is Tizanidine Hydrochloride. Tizanidine Hydrochloride in the present invention is used in an amount of about 2-40% by weight.

In another embodiment the present invention includes particle size of Tizanidine or its salt, wherein D₉₀ is less than about 200 μm and D₅₀ is less than about 80 μm.

In another embodiment of the invention, the pharmaceutical composition comprising Tizanidine Hydrochloride is prepared by using wet or dry granulation process. Any pharmaceutically acceptable granulating agent can be used for wet granulation. Suitable granulating agents include water, esters such as ethyl acetate; ketones such as acetone; alcohols such as methanol, ethanol, isopropanol, butanol; dichloromethane and combinations thereof. Preferably, the granulating agent used during wet granulation is water.

In another embodiment of the invention, wet granulation can be performed using Rapid mixer granulator, Fluid bed granulator, Planetary mixer and the like.

Another embodiment of the present invention encompasses a pharmaceutical composition comprising Tizanidine Hydrochloride and a pharmaceutically acceptable excipient selected from at least one of diluent and optionally binder, disintegrant, surfactant, lubricant and glidant.

Diluents or fillers are substances which usually provide bulk to the composition. Various useful fillers or diluents include, but are not limited to calcium phosphate (anhydrous or dihydrate), dibasic calcium phosphate, tribasic calcium phosphate, calcium carbonate, calcium sulfate, dextrates, dextrin, dextrose, fructose, kaolin, anhydrous lactose, lactose monohydrate, maltose, sugar alcohol (such as mannitol, sorbitol, xylitol, lactitol, maltitol, erythritol, isomalt etc.), sucrose, starch, pregelatinized starch, cellulose derivatives (microcrystalline cellulose etc) or talc. Preferably, filler is used in an amount of about 5-90% by weight. More preferably, filler is used in an amount of at least 50% by weight.

Binders impart cohesiveness to formulation. Various useful binders include, but are not limited to acacia, alginic acid, sodium alginate, carbomer, sodium carboxymethylcellulose, dextrin, ethylcellulose, gelatin, glucose, guar gum, hydroxypropylcellulose, hydroxypropylmethyl cellulose, maltose, methylcellulose, povidone, copovidone, starch, pregelatinized starch, modified starch, polyvinyl alcohol or polyethylene oxide. Preferably, binder is optionally used in amounts of about 0-10% by weight.

Various useful disintegrants include, but are not limited to, alginic acid, croscarmellose sodium, carmellose calcium, crospovidone, potassium polacrilin, sodium starch glycolate, low substituted hydroxypropyl cellulose or starch. Preferably, disintegrant is optionally used in amounts of about 0-10% by weight.

Lubricants are added to a pharmaceutical composition for ease in processing, to prevent adhesion to the equipment during processing. Lubricants used in the composition include lubricants commonly used in solid pharmaceutical compositions. Lubricants used in the composition include, but are not limited to, calcium stearate, magnesium stearate, mineral oil, glyceryl behenate, polyethylene glycol, sodium stearyl fumarate, stearic acid, talc, vegetable oil, sodium lauryl sulfate, silicon dioxide, carnauba wax, sucrose stearate or zinc stearate. Preferably, lubricant is present in an amount of about 0.1-5% by weight.

Glidants improve flowability and accuracy of dosing. Glidants used in the composition include, but are not limited to, colloidal silicon dioxide, magnesium trisilicate, starch, talc, or tribasic calcium phosphate. Preferably, the glidants are used in amounts of about 0.1-15% by weight.

In another embodiment of the invention, the pharmaceutical compositions are formulated into solid pharmaceutical dosage forms. Solid pharmaceutical dosage forms include, but are not limited to, tablets, capsules, powders, granules and sachets. Preferably, the solid pharmaceutical dosage form is a tablet or capsule. More preferably, the solid pharmaceutical dosage form is a capsule.

In yet another embodiment of the invention, the pharmaceutical compositions comprise about 2 mg to about 36 mg of Tizanidine Hydrochloride. Preferably, the pharmaceutical compositions comprise about 2 mg to about 18 mg of Tizanidine Hydrochloride. More preferably, the pharmaceutical compositions comprise about 2 mg to about 6 mg of Tizanidine Hydrochloride.

In another embodiment, at least 30% of the granules present in the pharmaceutical composition have a diameter of about 850 to about 250 μm [ASTM (American Society for Testing Materials) standards #20-60 mesh sieve]. Preferably, at least 40% the granules present in the pharmaceutical composition have a diameter of about 850 to about 250 μm [ASTM (American Society for Testing Materials) standards #20-60 mesh sieve]. More preferably, at least 50% the granules present in the pharmaceutical composition have a diameter of about 850 to about 250 μm [ASTM (American Society for Testing Materials) standards #20-60 mesh sieve].

Diameter of granules is determined using Retsch AS 200 magnetic sieve shaker at an amplitude of 30 to 90 Hz with time interval between 5 to 30 minutes (Refer: USP 29 <786> Particle size distribution estimation by analytical sieving).

In another embodiment, pharmaceutical compositions of the present invention are placed inside capsule shell of size 1 to 4.

In another embodiment, pharmaceutical compositions of the present invention exhibit more than 85% of drug release within 15 minutes in 500 ml of 0.01 N HCl in (Office of Generic Drugs dissolution database) using a USP II apparatus (paddle) at a temperature of 37±0.5° C. and a rotation speed of 50 revolutions per minute.

Another embodiment of the present invention also provides a process for the preparation of pharmaceutical composition of Tizanidine, comprising the steps of (a) blending a mixture of Tizanidine and at least one pharmaceutically acceptable excipient; (b) granulating the blend of step (a) with water; (c) drying the wet granules to obtain dried granules; (d) optionally milling of the dried granules; (e) adding at least one lubricant/glidant and optionally other pharmaceutically acceptable excipients to the dried milled granules; and (f) filling the lubricated granules in capsules.

The process for the invention besides being cost effective, also makes it possible to prepare a pharmaceutical composition of Tizanidine, wherein the composition has desirable formulation technical attributes.

Another embodiment of the present invention includes method of using the pharmaceutical composition comprising Tizanidine Hydrochloride in the management of spasticity.

As used herein, the term “about” means±approximately 20% of the indicated value, such that “about 10 percent” indicates approximately 08 to 12 percent.

### EXAMPLES

The following examples are intended to further illustrate certain preferred embodiments of the invention and are not limiting in nature.

### Example I

Tizanidine Hydrochloride capsules were prepared by wet granulation method by using quantitative formula as given in Table 1:

TABLE 1

| Ingredients | % (w/w) |
|---|---|
| Tizanidine Hydrochloride | 2.86 |
| Mannitol | 86.93 |
| Silicon dioxide | 2.08 |
| Hydroxypropyl methylcellulose | 3.13 |
| Talc | 5.0 |
| Purified Water | q.s. |

The processing steps involved in the manufacturing of capsules are given below:
i) Tizanidine, Mannitol and Silicon dioxide were sifted through a suitable sieve.
ii) The sifted blend of step i) was placed in a rapid mixer granulator and mixed for a suitable time.
iii) Binder solution was prepared by dissolving Hydroxypropyl methylcellulose in water.
iv) Blend of Step ii) was granulated using binder solution of step iii) in a rapid mixer granulator.
v) The granules of step iv) were dried in a fluidized bed dryer.
vi) The dried granules were milled and sized using suitable sieve.
vii) Granules obtained from step vi) were lubricated with Talc and filled into hard gelatin capsules.

### Example II

Tizanidine Hydrochloride capsules were prepared by wet granulation method by using quantitative formula as given in Table 2:

TABLE 2

| Ingredients | % (w/w) |
|---|---|
| Tizanidine Hydrochloride | 3.1 |
| Lactose | 74.2 |
| Silicon dioxide | 1.4 |
| Microcrystalline cellulose | 10.7 |
| Hydroxypropyl methylcellulose | 5.3 |
| Talc | 5.3 |
| Purified Water | q.s. |

The processing steps involved in the manufacturing of capsules are given below:
i) Tizanidine, Lactose, Microcrystalline cellulose and Silicon dioxide were sifted through a suitable sieve.
ii) The sifted blend of step i) was placed in a fluid bed processor and mixed.
iii) Binder solution was prepared by dissolving Hydroxypropyl methylcellulose in water.
iv) Blend of Step ii) was granulated by spraying binder solution of step iii) into a fluid bed processor.
v) The granules of step iv) were dried in a fluidized bed dryer.
vi) The dried granules were milled and sized using suitable sieve.
vii) Granules obtained from step vi) were lubricated with Talc and filled into hard gelatin capsules.

### Example III

The standardized method and equipment for testing dissolution time is provided in Office of Generic Drugs dissolution database. The dissolution profile of capsules prepared using quantitative composition as given in Table 1 and Table 2 was measured in 500 ml of 0.01 N HCl in (Office of Generic Drugs dissolution database) using a USP II apparatus (paddle) at a temperature of 37±0.5° C. and a rotation speed of 50 revolutions per minute. The dissolution test was conducted on the reference formulation ZANAFLEX® capsules in comparison to a capsule dosage form as given in Example 1 and Example 2. Since, both commercially available ZANAFLEX® capsules and capsules prepared using quantitative composition as given in Table 1 and Table 2 exhibited more than 85% of drug release within 15 minutes, dissolution profiles of two formulations were found to be similar.

### Example IV

Capsule dosage forms prepared in Example I and Example II were subjected to Accelerated stability testing as per the ICH guidelines at temperature/relative humidity of 40°±2° C./75%±5% RH for 3 months by High Performance Liquid Chromatography (HPLC) method. The prepared dosage form was found to be stable and exhibited following assay values (in the Table 3):

TABLE 3

| | Amount of Tizanidine in the Capsule dosage form | |
|---|---|---|
| Study period | Example I | Example II |
| Initial | 101.7% | 99.8% |
| After one month | 101.7% | 99.2% |
| After three months | 101.7% | 100% |

Many modifications of this invention can be made without departing from its spirit and scope, as will be evident to those skilled in the art. The specific embodiments described herein are provided by way of example only, and the invention is to be limited only by the terms of the appended claims, along with the full scope of equivalents to which such claims are entitled.

1. A pharmaceutical capsule dosage form comprising Tizanidine or its pharmaceutically acceptable salts, esters, solvates, polymorphs, stereoisomers or mixtures thereof, wherein the dosage form is free of non-pareil seeds.

2. A pharmaceutical capsule dosage form comprising Tizanidine or its pharmaceutically acceptable salts, esters, solvates, polymorphs, stereoisomers or mixtures thereof, wherein the dosage form is free of non-pareil seeds and comprises at least 30% of the granules with a diameter of about 850 to about 250 μm (ASTM #20-60 mesh sieve).

3. The pharmaceutical capsule dosage form of claim 2, wherein at least 55% of the granules present in the dosage form have a diameter of about 850 to about 250 μm (ASTM #20-60 mesh sieve).

4. The pharmaceutical capsule dosage form as claimed in claim 1, wherein the dosage form is prepared by dry granulation.

5. The pharmaceutical capsule dosage form as claimed in claim 1, wherein the dosage form is prepared by wet granulation method.

6. The pharmaceutical capsule dosage form of claim 5, wherein the dosage form comprises at least one or more pharmaceutically acceptable excipient.

7. The pharmaceutical dosage form of claim 6, wherein the pharmaceutically acceptable excipient is selected from diluent, binder, disintegrant, surfactant, lubricant, and glidant.

8. The pharmaceutical dosage form as claimed in claim 1, wherein Tizanidine or its pharmaceutically acceptable salts, esters, solvates, polymorphs, stereoisomers or mixtures thereof is present in an amount ranging from about 2 mg to about 36 mg.

9. A pharmaceutical capsule dosage form comprising Tizanidine or its pharmaceutically acceptable salts, esters, solvates, polymorphs, stereoisomers or mixtures thereof, wherein the dosage form is free of non-pareil seeds and comprises:
a) at least 30% of the granules present in the capsule dosage form with a diameter of about 850 to about 250 μm (ASTM #20-60 mesh sieve),
b) particle size of Tizanidine where D90 is less than about 100 μm.

10. A process for the preparation of a pharmaceutical capsule dosage form of Tizanidine free from non-pareil seeds, wherein the process comprises:
(a) blending a mixture of Tizanidine and at least one pharmaceutically acceptable excipient; Optionally
(b) granulating the blend of step (a) with water;
(c) drying the wet granules;
(d) milling and sizing the dried granules, followed by adding at least one lubricant/glidant and optionally other pharmaceutically acceptable excipients to the dried milled granules; and
(e) filling lubricated granules in capsules.
